(19) United States
(12) Patent Application Publication
(10) Pub. No.: US 2010/0292781 A1
(43) Pub. Date: Nov. 18, 2010

(54) STENT ADVANCEMENT ASSISTANT AND LESION DILATOR WIRE

(76) Inventors: Gurpreet S. Sandhu, Rochester, MN (US); Charanjit S. Rihal, Rochester, MN (US); Rajiv Gulati, Rochester, MN (US); John F. Bresnahan, Rochester, MN (US)

Correspondence Address: FISH & RICHARDSON P.C., PO BOX 1022, MINNEAPOLIS, MN, 55440-1022, US

(21) Appl. No.: 12/812,530

(22) PCT Filed: Jan. 5, 2009
(86) PCT No.: PCT/US09/30094

§ 371 Date: Jul. 12, 2010

Related U.S. Application Data
(60) Provisional application No. 61/020,503, filed on Jan. 11, 2008.

(51) Int. Cl. A61F 2/82

(52) U.S. Cl. 623/1.23

(57) ABSTRACT

Materials and methods for placement of catheters, stents, and other therapeutic or diagnostic devices in blood vessels.

### CROSS-REFERENCE TO RELATED APPLICATIONS

This application claims priority from U.S. Provisional Application Ser. No. 61/020,503, filed Jan. 11, 2008.

### TECHNICAL FIELD

This document relates to materials and methods for widening or opening narrowed or obstructed blood vessels, and for placement of stents and wires in blood vessels.

### BACKGROUND

Angioplasty is the mechanical widening of a narrowed or totally obstructed blood vessel. These obstructions often are caused by atherosclerosis. The term “angioplasty” has come to include all manner of vascular interventions typically performed in a minimally-invasive or “percutaneous” method.

Coronary angioplasty, also referred to as percutaneous coronary intervention, is a therapeutic procedure to treat stenotic (narrowed) coronary arteries of the heart found in coronary heart disease. These stenotic segments result from the build-up of cholesterol-laden plaques that form due to atherosclerosis. Coronary angioplasty typically is performed by an interventional cardiologist.

Peripheral angioplasty refers to the use of mechanical widening to open blood vessels other than the coronary arteries. This procedure often is referred to as percutaneous transluminal angioplasty, or PTA. PTA is most commonly performed to treat narrowing in the leg arteries, especially the common iliac, external iliac, superficial femoral, and popliteal arteries. PTA also can be used to treat narrowing of veins. In addition, atherosclerotic obstruction of the renal artery can be treated with angioplasty of the renal artery (percutaneous transluminal renal angioplasty, PTRA). Carotid artery stenosis also can be treated with angioplasty.

Any of these angioplasty procedures can include placement of a stent to prevent or counteract a disease-induced constriction of localized blood flow. The stent can act as a scaffold, remaining in place permanently to help keep the vessel open. A stent typically is inserted through a main artery in the groin (femoral artery) or arm (brachial artery) on a wire or catheter (e.g., a balloon catheter, in the case of balloon angioplasty), and threaded up to the narrowed section of the vessel. In a balloon angioplasty procedure, the balloon can be inflated to push the plaque out of the way and expand the vessel. In a balloon angioplasty/stent placement procedure, a stent either can be stretched open by the balloon at the same time as the artery, or can be inserted into the vessel immediately after the angioplasty procedure. Once in place, the stent helps to hold the vessel open, thus improving blood flow.

### SUMMARY

Coronary and peripheral vessels that require angioplasty and stenting often contain previously placed stents and/or calcified or hardened plaques, which can interfere with placement of a new stent. The distal tip of a stent catheter or the tines on a stent, for example, can drag on or become hooked against the obstruction, making it difficult to advance the stent to its desired location. Some procedures have included the use of two wires, one stiff and the other floppy, which both are placed in the vessel. The stent then can be advanced by trial and error over one or the other wire and “jiggled” across the obstruction. Other procedures have included the use of three wires—two of which can form a track or groove, and the third of which can be used to advance the stent along the groove formed by the first two wires. These procedures have had limited success, however, and improved methods are needed.

This document is based, in part, on the discovery that a wire having a soft, atraumatic, silastic slide can be used as a deflector during stent placement. Such a wire can be positioned across an obstruction in a blood vessel, whereupon the narrowed circumference of the vessel can force the silastic slide to fold inward and form one or more channels along which the stent catheter and stent (on a separate placement wire) can slide. Procedures using such a deflector wire can successfully and reproducibly place stents over calcified plaques and old stents.

This document also is based, in part, on the discovery that a wire having a region of graduated diameter (a “dilator wire”) can be used to open or widen a passage through a blockage in a blood vessel. Such a wire can have a segment that gradually widens and then gradually tapers. Advancement of this segment through an obstructed region of a blood vessel can open a passage for an angioplasty catheter and/or a stent, for example, and thus can facilitate angioplasty and stent placement procedures. The channel created by advancement of a dilator wire also can allow subsequent contrast dye and blood passage into the distal vessel, which in some cases remains totally occluded after a standard guide wire is advanced across a critically tight blockage.

In one aspect, this document features a method for placing a device into a blood vessel, comprising advancing into the blood vessel a first wire having a distal end and a flexible silastic member, wherein the flexible silastic member is positioned proximate the distal end. The first wire can comprise an elongate relatively rigid portion and a relatively floppy portion, the floppy portion being at the distal end of the wire, wherein the flexible silastic member is positioned on the rigid portion proximate the floppy portion. The flexible silastic member can be about 1 cm to about 3 cm proximal to the floppy portion (e.g., about 2 cm proximal to the floppy portion). The flexible silastic member can comprise silicone, polyethylene terephthalate, polytetrafluorethylene, or a plastic or polymer coated with TEFLON®. The flexible silastic member can have a length of about 10 mm to about 25 mm and a width of about 1.5 mm to about 5 mm. The flexible silastic member can be flat when in an uncompressed configuration. The first wire can be about 120 cm to about 210 cm in length and about 0.01 inch to about 0.4 inch in diameter. The method can further comprise advancing the flexible silastic member to the location of an obstruction in the blood vessel, and advancing a catheter and/or stent through the blood vessel such that a portion of the catheter and/or stent moves along the flexible silastic member. The obstruction can be a plaque or a previously placed stent. The catheter can be a balloon catheter with a stent positioned circumferentially around the balloon. The method can further comprise at least partially withdrawing the first wire from the blood vessel, and inflating the balloon to expand the stent.

In another aspect, this document features a wire comprising a distal end and a flexible silastic member, wherein the flexible silastic member is positioned proximate the distal end. The wire can comprise an elongate rigid portion and a floppy portion, the floppy portion being at the distal end of the wire, wherein the flexible silastic member is positioned on the rigid portion proximate the floppy portion. The flexible silastic member can be about 1 cm to about 3 cm proximal to the floppy portion (e.g., about 2 cm proximal to the floppy portion). The flexible silastic member can comprise silicone, polyethylene terephthalate, polytetrafluorethylene, or a plastic or polymer coated with TEFLON®. The flexible silastic member can have a length of about 10 mm to about 25 mm and a width of about 1.5 mm to about 5 mm. The flexible silastic member can be flat when in an uncompressed configuration. The wire can be about 120 to about 210 cm in length and about 0.01 inch to about 0.4 inch in diameter.

In another aspect, this document features an elongate wire having a distal end, wherein a segment of the wire proximate the distal end has a diameter that gradually increases and then gradually decreases. The wire can be comprised primarily of an elongate relatively rigid portion, and, in some embodiments, also can have a relatively floppy portion at the distal end. The segment of graduated diameter can be about 1 cm to about 3 cm in length (e.g., about 2 cm in length), and can be located about 1 cm to about 3 cm (e.g., about 2 cm) from the distal end of the wire, or about 1 cm to about 3 cm (e.g., about 2 cm) from the junction between the relatively rigid portion and the relatively floppy portion. The diameter of the wire at its narrowest portion can be about 0.01 inch to about 0.02 inch. The segment of graduated diameter can increase in size to a diameter of about 0.02 to about 0.04 inch at its widest portion.

In still another aspect, this document features a method for placing a device into a blood vessel, comprising advancing into the blood vessel a first wire having a distal end and a region of graduated diameter proximate the distal end, the region of graduated diameter comprising a widest portion. The wire can be comprised primarily of an elongate relatively rigid portion, and, in some embodiments, also can have a relatively floppy portion at the distal end. The segment of graduated diameter can be about 1 cm to about 3 cm in length (e.g., about 2 cm in length), and can be located about 1 cm to about 3 cm (e.g., about 2 cm) from the distal end of the wire, or about 1 cm to about 3 cm (e.g., about 2 cm) from the junction between the relatively rigid portion and the relatively floppy portion. The diameter of the wire at its narrowest portion can be about 0.01 inch to about 0.02 inch. The segment of graduated diameter can increase in size to a diameter of about 0.02 to about 0.04 inch at its widest portion. The method can further comprise advancing the wire into the blood vessel until the widest portion passes through an obstruction. The method can further comprise passing a balloon catheter into the blood vessel (e.g., over the wire).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims

### DESCRIPTION OF DRAWINGS

FIG. 1 is a longitudinal cross-sectional view of a blood vessel having an obstruction, with a wire and a stent positioned therein.

FIG. 2 is a longitudinal cross-sectional view of a blood vessel having an obstruction, with a deflector wire and a wire and stent positioned therein.

FIGS. 3A and 3B are lateral cross-sectional view of a blood vessel having a deflector wire therein. FIG. 3A shows a portion of the blood vessel where there is no obstruction, while FIG. 3B shows a portion of the blood vessel where there is an obstruction.

FIG. 4 is a longitudinal cross-sectional view of a blood vessel having an obstruction, with a wire having a graduated diameter positioned therein.

Like reference symbols in the various drawings indicate like elements.

### DETAILED DESCRIPTION

This document provides methods for facilitating advancement of a stent, balloon, or another therapeutic or diagnostic device across an obstruction in a blood vessel. The obstruction can be, for example, a plaque (e.g., a hardened or calcified plaque), or a previously placed stent.

In some embodiments, the methods provided herein can include using a wire having a soft, atraumatic slide (e.g., a silastic silicone slide) as a deflector to facilitate advancement of a stent, balloon, or other diagnostic or therapeutic device (e.g., an intravascular ultrasound or a filter wire). Such a deflector wire can be advanced into a blood vessel such that the slide is positioned across an obstruction, whereupon the narrowed circumference of the vessel can force the slide to flex or fold inward, thus forming one or more channels along which the first wire can slide.

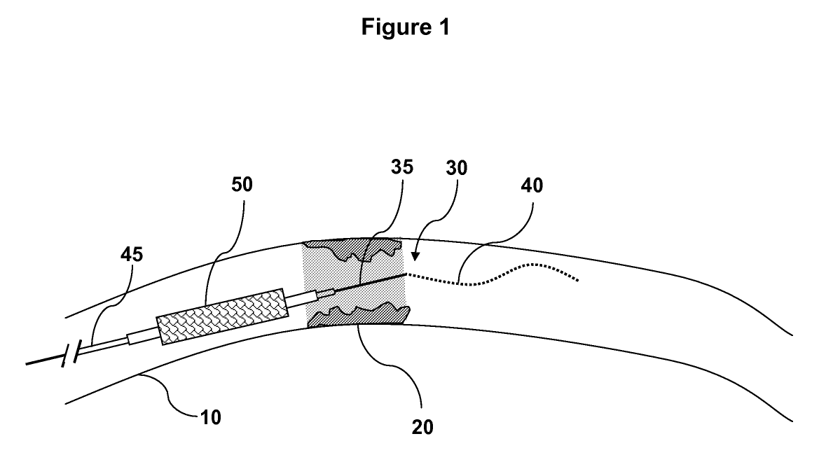

As depicted in FIG. 1, blood vessel 10 can have an obstruction such as plaque 20. Guide wire 30 can be advanced through blood vessel 10 and across plaque 20. In some embodiments, wire 30 can be a standard angioplasty wire. For example, wire 30 can be comprised primarily of relatively stiff (but still flexible) portion 35, with floppy portion 40 at its distal end. Wire 30 can be, for example, a guide wire for catheter 45, such that catheter 45 is positioned over wire 30 either before or after advancement of wire 30 into vessel 10. In some embodiments, catheter 45 can be a balloon catheter. In addition, stent 50 can be positioned circumferentially around catheter 45, e.g., around a portion of catheter 45 that is toward the distal end of stiff portion 35 of wire 30. During an angioplasty and/or stent placement procedure, the distal tip of catheter 45 or stent 50 can snag or catch on obstruction 20, thus hindering or even prohibiting placement of catheter 45 and/or stent 50.

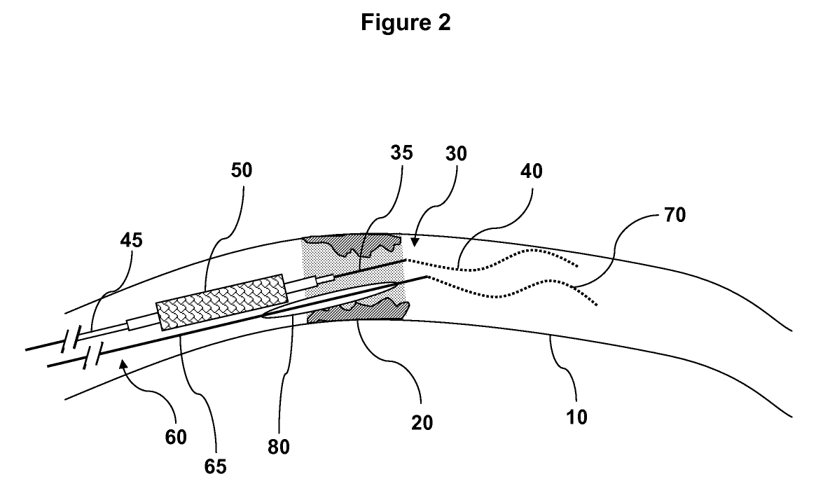

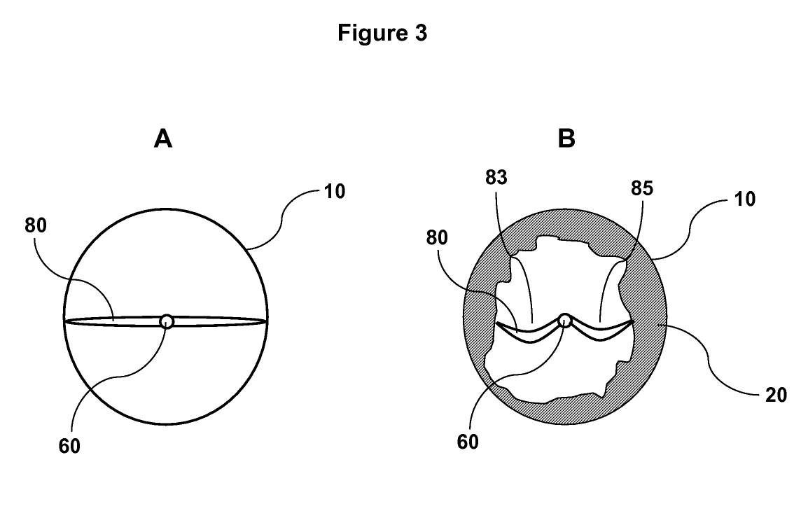

To circumvent such problems, second wire 60 also can be placed in vessel 10 and advanced to plaque 20 as shown in FIG. 2, for example. Wire 60 can be comprised primarily of relatively stiff (but still flexible) portion 65, with floppy portion 70 at its distal end. Wire 60 also can have slide 80, which can serve as a deflector for wire 30 and/or stent 50. As depicted in FIG. 3A, for example, slide 80 can be relatively flat when in its uncompressed configuration. When slide 80 is advanced to an obstruction such as plaque 20, however, the reduced diameter of vessel 10 can force slide 80 to flex, thus forming channels 83 and 85 (FIG. 3B). Wire 30 can be advanced along channel 83 and/or channel 85, thus permitting placement of wire 30 and/or stent 50 across the obstruction.

Wire 30 and wire 60 can have any suitable dimensions and can comprise any suitable material. For example, wire 30 and wire 60 can be about 120 to about 210 cm in length (e.g., about 120 to about 130 cm, about 150 to about 160, or about 190 to about 210 cm in length). Floppy portion 70 of wire 60 can be about 1 to about 4 cm in length (e.g., about 1 cm, about 1.5 cm, about 2.0 cm, about 2.5 cm, about 3 cm, about 3.5 cm, or about 4 cm in length). Wire 60 can have a diameter of about 0.010 inch to about 0.4 inch (e.g., 0.014 inch, 0.02 inch, 0.025 inch, 0.03 inch, 0.05 inch, 0.08 inch, 0.1 inch, 0.15 inch, 0.2 inch, 0.25 inch, 0.3 inch, 0.35 inch, or 0.38 inch). Slide 80 can be about 1.5 mm to about 5 mm wide (e.g., about 1.5 mm, about 1.7 mm, about 2 mm, about 2.3 mm, about 2.5 mm, about 2.8 mm, about 3 mm, about 3.2 mm, about 3.5 mm, about 3.7 mm, about 4 mm, about 4.3 mm, about 4.5 mm, about 4.8 mm, or about 5 mm wide). Slide 80 can have a length of about 10 mm to about 25 mm (e.g., about 10 mm, about 12 mm, about 15 mm, about 17 mm, about 20 mm, about 22 mm, or about 25 mm). Wire 60 also can have any degree of flexibility. For example, floppy portion 70 of wire 60 can have a flexibility of about 2.5 gm to about 5 gm (e.g., about 2.5 gm, about 3 gm, about 4 gm, or about 5 gm), and relatively stiff portion 65 of wire 60 can have a flexibility from about 6 gm to about 12 gm (e.g., about 6 gm, about 9 gm, or about 12 gm). The wires can comprise materials such as, for example, metals or alloys of metals (e.g., steel, nitinol, or cobalt-chromium). In some embodiments, a wire can comprise a plastic or polymer material, (e.g., as a tip or a coating). Some wires can have radio-opaque markers containing gold or platinum, for example.

Slide 80 can be a permanently molded onto wire 60, and can be positioned about 1 cm to about 3 cm proximal to floppy portion 70, such that there is about 1 cm to about 3 cm (e.g., about 1 cm about 1.2 cm, about 1.5 cm, about 1.8 cm, about 2 cm, about 2.3 cm, about 2.5 cm, about 2.8 cm, or about 3 cm) of space between the distal end of slide 80 and the junction between relatively rigid portion 65 and floppy portion 70 of wire 60. Slide 80 can comprise any suitable material. For example, slide 80 typically comprises a pliable, slippery substance such as silicone, polyethylene terephthalate (PET), polytetrafluorethylene (PTFE), or a plastic or polymer coated with TEFLON®. Slide 80 can be of a material that naturally tries to unfold in a flat plane, thus avoiding obstruction of blood flow when placed into a blood vessel.

During an angioplasty procedure, guide wire 30 can be advanced into vessel 10 such that at least a portion of floppy portion 40 is positioned across an obstruction such as plaque 20. Wire 60 then can be advanced into vessel 10 such that slide 80 is positioned over plaque 20, causing slide 80 to flex and form channels 83 and 85. Wire 30, catheter 45, and/or stent 50, subsequently can be advanced over slide 80 and across the obstruction. Once wire 30, catheter 45, and/or stent 50 are positioned over the obstruction, wire 60 can be partially or fully retracted from vessel 10, or can remain in position across the obstruction during, for example, an angioplasty procedure. During a procedure in which a stent is expanded at the site of the obstruction, it may be desirable to at least partially retract wire 60 from vessel 10 prior to expansion of the stent. It is noted, however, that in addition to serving as a deflector for catheter 45 and/or stent 50, slide 80 can be used to hold an intimal dissection flap in place while stent 50 is being placed. Once the angioplasty and/or stent placement procedure is completed, wire 60 can be advanced further into vessel 10 for additional procedures, if desired.

In some embodiments, the methods provided herein can include using a wire having a graduated diameter to open or widen a hole through an obstruction (e.g., a chronic total occlusion) in a blood vessel. After the widest portion of the wire has been passed through an obstructed section of the vessel, a balloon catheter or stent can be more readily advanced to the site. A wire having a graduated diameter can be used with or without a deflector wire as described herein.

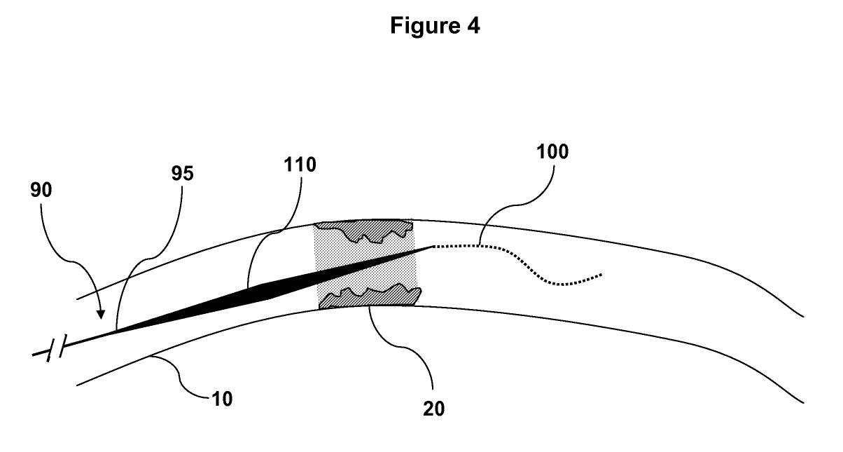

As depicted in FIG. 4, for example, wire 90 can be advanced into vessel 10 toward plaque 20. Wire 90 can have relatively rigid (but still flexible) portion 95, and, in some embodiments, can have floppy portion 100. Wire 90 can be similar to a standard interventional guide wire, but can include graduated section 110 at or near its distal end, which gradually increases in diameter and then tapers to a smaller diameter. Section 110 can be about 1 cm to about 3 cm in length (e.g., about 1, about 1.5, about 2, about 2.5, or about 3 cm in length), and can be located about 1 cm to about 3 cm (e.g., about 1, about 1.5, about 2, about 2.5, or about 3 cm) from the distal end of wire 90, or about 1 cm to about 3 cm (e.g., about 1, about 1.5, about 2, about 2.5, or about 3 cm) from the junction between rigid portion 95 and floppy portion 100. The diameter of wire 90 at its narrowest portion can be about 0.01 inch (e.g., about 0.01 inch, about 0.014 inch, about 0.015 inch, about 0.017 inch, or about 0.02 inch). Section 110 can increase in size to a diameter of about 0.02 to about 0.04 inch (e.g., about 0.02 inch, about 0.025 inch, about 0.03 inch, about 0.032 inch, about 0.035 inch, about 0.038 inch, or about 0.04 inch) at its widest portion.

In use, wire 90 can be advanced into vessel 10 until the widest portion of section 110 passes through an obstruction such as plaque 20. In some embodiments, a balloon catheter then can be passed over wire 90 for an angioplasty procedure. The catheter also can have a stent positioned thereon, for placement at the site of the obstruction. In other embodiments, a separate guide wire, with or without a catheter and stent, can be advanced into the vessel after section 110 of wire 90 has been advanced through the obstruction. In some of these cases, a deflector wire such as that depicted in FIGS. 2 and 3 can be advanced into the vessel along with or prior to the guide wire.

In some embodiments, wire 90 and wire 60 essentially can be combined, such that the wire has a section of graduated diameter located toward the distal end, and a silastic slide located proximal to the section of graduated diameter. In such embodiments, the wire can be advanced into a blood vessel such that the section of graduated diameter passes through and opens or widens a hole in an obstruction (e.g., a calcified or hardened plaque), and then can be further advanced until the slide is located at the site of the obstruction. The remaining obstruction can force the slide to flex inward, forming one or more channels along which a separate guide wire (e.g., wire 30), with or without a catheter and a stent, can be advanced.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for placing a device into a blood vessel, comprising advancing into the blood vessel a first wire having a distal end and a flexible silastic member, wherein the flexible silastic member is positioned proximate the distal end.

2. The method of claim 1, wherein the first wire comprises an elongate rigid portion and a floppy portion, the floppy portion being at the distal end of the wire, wherein the flexible silastic member is positioned on the rigid portion proximate the floppy portion.

3. The method of claim 2, wherein the flexible silastic member is about 1 cm to about 3 cm proximal to the floppy portion.

4. The method of claim 2, wherein the flexible silastic member is about 2 cm proximal to the floppy portion.

5. The method of claim 1, wherein the flexible silastic member comprises silicone, polyethylene terephthalate, polytetrafluorethylene, or a plastic or polymer coated with TEFLON®.

6. The method of claim 1, wherein the flexible silastic member has a length of about 10 mm to about 25 mm and a width of about 1.5 mm to about 5 mm.

7. The method of claim 1, wherein the flexible silastic member is flat when in an uncompressed configuration.

8. The method of claim 1, wherein the first wire is about 120 cm to about 210 cm in length and about 0.01 inch to about 0.4 inch in diameter.

9. The method of claim 1, further comprising advancing the flexible silastic member to the location of an obstruction in the blood vessel, and advancing a catheter and/or stent through the blood vessel such that a portion of the catheter and/or stent moves along the flexible silastic member.

10. The method of claim 9, wherein the obstruction is a plaque or a previously placed stent.

11. The method of claim 9, wherein the catheter is a balloon catheter with a stent positioned circumferentially around the balloon.

12. The method of claim 11, further comprising at least partially withdrawing the first wire from the blood vessel, and inflating the balloon to expand the stent.

13. A wire comprising a distal end and a flexible silastic member, wherein the flexible silastic member is positioned proximate the distal end.

14. The wire of claim 13, wherein the wire comprises an elongate rigid portion and a floppy portion, the floppy portion being at the distal end of the wire, wherein the flexible silastic member is positioned on the rigid portion proximate the floppy portion.

15. The wire of claim 14, wherein the flexible silastic member is about 1 cm to about 3 cm proximal to the floppy portion.

16. The wire of claim 14, wherein the flexible silastic member is about 2 cm proximal to the floppy portion.

17. The wire of claim 13, wherein the flexible silastic member comprises silicone, polyethylene terephthalate, polytetrafluorethylene, or a plastic or polymer coated with TEFLON®.

18. The wire of claim 13, wherein the flexible silastic member has a length of about 10 mm to about 25 mm and a width of about 1.5 mm to about 5 mm.

19. The wire of claim 13, wherein the flexible silastic member is flat when in an uncompressed configuration.

20. The wire of claim 13, wherein the wire is about 120 to about 210 cm in length and about 0.01 inch to about 0.4 inch in diameter.
